(19) United States
(12) Patent Application Publication
(10) Pub. No.: US 2024/0395381 A1
(43) Pub. Date: Nov. 28, 2024

(54) Interactive PREP Dispensers and Methods of Using Interactive PREP Dispensers

(71) Applicant: Rachel Rivera, Dallas, TX (US)

(72) Inventor: Rachel Rivera, Dallas, TX (US)

(21) Appl. No.: 18/200,014

(22) Filed: May 22, 2023

(51) Int. Cl. G16H 20/13; G16H 10/40

(52) U.S. Cl. CPC G16H 20/13; G16H 10/40

(57) ABSTRACT

An interactive medication dispenser is disclosed, and more specifically interactive PrEP dispensers and methods of using interactive PrEP dispensers are disclosed for providing out of clinic PrEP prescriptions and dispensing. A system for remote interactive dispensing of PrEP is disclosed. The system having a kiosk, a user device, and a provider device in network communication with each other. Methods of using an interactive PrEP dispenser and a system for dispensing PrEP are disclosed.

### TECHNICAL FIELD

This application is directed, in general, to an interactive medication dispenser, and more specifically to interactive PrEP dispensers and methods of using interactive PrEP dispensers.

### BACKGROUND

In 2021, there were about 1.5 million new cases of HIV in 2021. New cases of HIV can be prevented or reduced by the use of pre-exposure prophylaxis (PrEP). When used as prescribed PrEP medications are considered highly effective for preventing HIV. PrEP can reduce the transmission of HIV from sex or injection drug use.

Currently, there are two FDA approved oral PrEP medications, which are Truvada and Descovy. Truvada is a combination of emtricitabine and tenofovir disoproxil fumarate. Descovy is a combination of emtricitabine and tenofovir alafenamide. Apretude is another FDA approved PrEP medication. However, Apretude is approved for administration by injection.

Oral PrEP medications may be used in a maintenance manner for individuals who are at an ongoing risk of contracting HIV, such as people who routinely have sex with an individual who is HIV positive. Maintenance dosing of PrEP typically includes ongoing repeated doses of PrEP medication. PrEP medications have also been shown to be effective in preventing HIV when dosed on “on demand.” On demand dosing refers to the use of PrEP prior to conduct that might result in HIV transmission. On demand dosing is also referred to as “intermittent,” “non-daily,” “event driven,” and “off-label” PrEP use.

A common on demand dosing of PrEP is the “2-1-1” schedule. In the 2-1-1 schedule, a person takes 2 PrEP pills 2-24 hours before sex, which poses a risk of transmitting HIV; 1 PrEP pill 24 hours after the first dose, and 1 PrEP 24 hours after the second dose. This dosing method has been shown to effective in preventing HIV transmission in gay and bisexual men who have anal sex without a condom. In some circumstances, on demand PrEP dosing is considered an effective alternative to daily PrEP dosages.

Prior to being prescribed PrEP and taking PrEP, whether using a maintenance dose plan or on demand dosing, an individual must meet certain requirements based on the drug being used. For example, current PrEP drugs should not be used by people who are currently HIV positive. In addition, PrEP drugs can only be prescribed by medical personnel that are approved to write PrEP prescriptions.

One common issue involving the use of PrEP to prevent HIV transmission, relates to access and availability of PrEP to individuals that may benefit from the use of PrEP. Typically, prior to receiving an initial PrEP dose, an individual must make an appointment with a health care provider and conduct testing and diagnosis to determine if PrEP is appropriate and fill a prescription at a pharmacy. This is most often done in person, which requires people to take the time required to obtain PrEP. Phone and mail in tests are another possible route to obtaining PrEP. However, this requires even more time to process and for a person to obtain PrEP doses. The same is true for obtaining PrEP prescription refills. This makes it harder for people to access and use PrEP medications, which results in the possible further spread of HIV.

These access and availability issues are even more significant when for people who do not desire to take daily doses of PrEP medications and instead prefer on demand PrEP use. This may include people who only occasionally engage in sexual intercourse with the possibility of transmitting HIV. These individuals have less motivation to go through the steps to obtain PrEP medications prior to engaging in such sex.

The need for quick and easy access to PrEP medications is particularly emphasized in situations where a person only occasionally engages in sexual activity that is likely to lead to the transmission of HIV. Such a person is not likely to have a PrEP maintenance dosage routine or even to have PrEP available for on demand dosing. If such a person spontaneously decides to engage in sexual activity that is likely to transmit HIV, that person would be unable to obtain a PrEP prescription prior to engaging in that sexual activity if that person was required to schedule and attend a medical provider appointment to obtain a PrEP prescription.

### BRIEF SUMMARY OF THE INVENTION

Features of an interactive PrEP dispenser, in one embodiment, includes a dispenser kiosk with a user interaction module with user interactive components such as a display screen, which may be a touch screen, and buttons for the user to interact with the dispenser. In addition, the kiosk has one or more dispensers for HIV test kit, PrEP medication, and condom and lubrication dispensing. The kiosk also has a user identification module with identification components for identifying the user, such as an integrated camera, an identification scanner, and/or a facial recognition software. The kiosk also has communication components used to communicate with a medical provider capable of prescribing PrEP, which allows for remote communication with the medical provider for transmitting information between the kiosk, the user, and the medical provider, such as transmitting test result and other information. The communication components may be typical network components such as a wired or wireless internet connection. In addition, the communication components of the kiosk may include the ability to communicate with a cell phone of a user or a medical provider through a cellular or other connection, such as a Blue Tooth connection. User interactions with the kiosk and the health provider or the user may be through a specific application operated on a cellular phone. The kiosk also has an HIV test reader.

A user desiring to obtain PrEP from a PrEP dispenser goes through the steps of 1) initiating a request for PrEP through the kiosk or application on the user's cellular phone; 2) obtaining information regarding the user's identity and identity verification; 3) obtaining information about the user regarding the user's qualification for PrEP medication; 4) conducting a user HIV test; 5) transmitting collected information, including user identification information, user qualification information, and HIV test results to a health provider; 6) making a prescribing determination; and 7) dispensing from the kiosk to the user based on the prescribing determination.

### DESCRIPTION OF THE DRAWINGS

Illustrative embodiments of the present invention are described in detail below with reference to the attached drawing figures, which are incorporated by reference herein and wherein:

FIG. 1A is a schematic representation of an embodiment of a system for remote interactive PrEP dispensing of PrEP.

FIG. 1B is a schematic view of an embodiment of an interactive PrEP dispenser.

FIG. 2 is a schematic description of a method for use of an interactive PrEP dispenser or a system for remote interactive PrEP dispensing of PrEP.

### DETAILED DESCRIPTION

In the following detailed description of the preferred embodiments, reference is made to the accompanying drawings that form a part hereof, and in which is shown, by way of illustration, specific embodiments in which the invention may be practiced. These embodiments are described in sufficient detail to enable those skilled in the art to practice the invention, and it is understood that other embodiments may be utilized and that logical structural, mechanical, electrical, and chemical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the claims.

Unless otherwise indicated, as used throughout this document, “or” does not require mutual exclusivity.

Features of an interactive PrEP dispenser, in one embodiment, includes a dispenser kiosk with a user interaction module with user interactive components such as a display screen, which may be a touch screen, and buttons for the user to interact with the dispenser. In addition, the kiosk has one or more dispensers for HIV test kit, PrEP medication, and condom and lubrication dispensing. The kiosk also has a user identification module with identification components for identifying the user, such as an integrated camera, an identification scanner, and/or a facial recognition software. The kiosk also has communication components used to communicate with a medical provider capable of prescribing PrEP, which allows for remote communication with the medical provider for transmitting information between the kiosk, the user, and the medical provider, such as transmitting test result and other information. The communication components may be typical network components such as a wired or wireless internet connection. In addition, the communication components of the kiosk may include the ability to communicate with a cell phone of a user or a medical provider through a cellular or other connection, such as a Blue Tooth connection. User interactions with the kiosk and the health provider or the user may be through a specific application operated on a cellular phone. The kiosk also has an HIV test reader.

A user desiring to obtain PrEP from a PrEP dispenser goes through the steps of 1) initiating a request for PrEP through the kiosk or application on the user's cellular phone; 2) obtaining information regarding the user's identity and identity verification; 3) obtaining information about the user regarding the user's qualification for PrEP medication; 4) conducting a user HIV test; 5) transmitting collected information, including user identification information, user qualification information, and HIV test results to a health provider; 6) making a prescribing determination; and 7) dispensing from the kiosk to the user based on the prescribing determination.

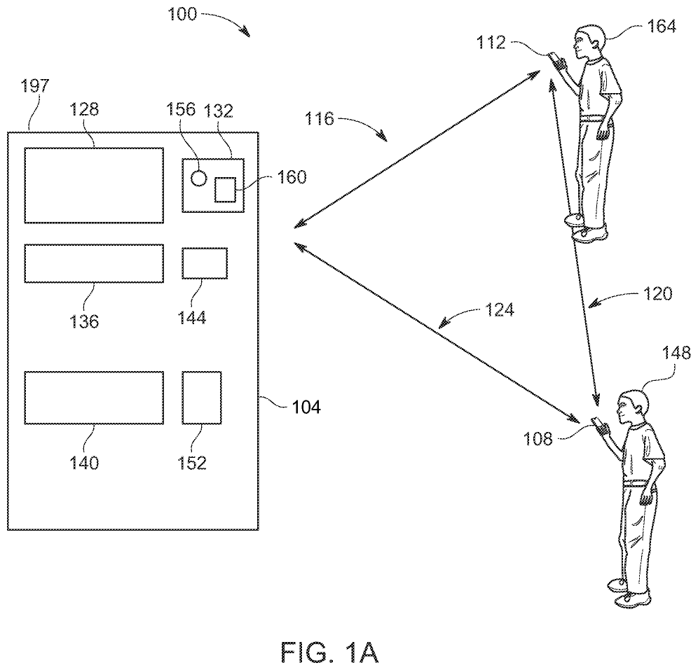

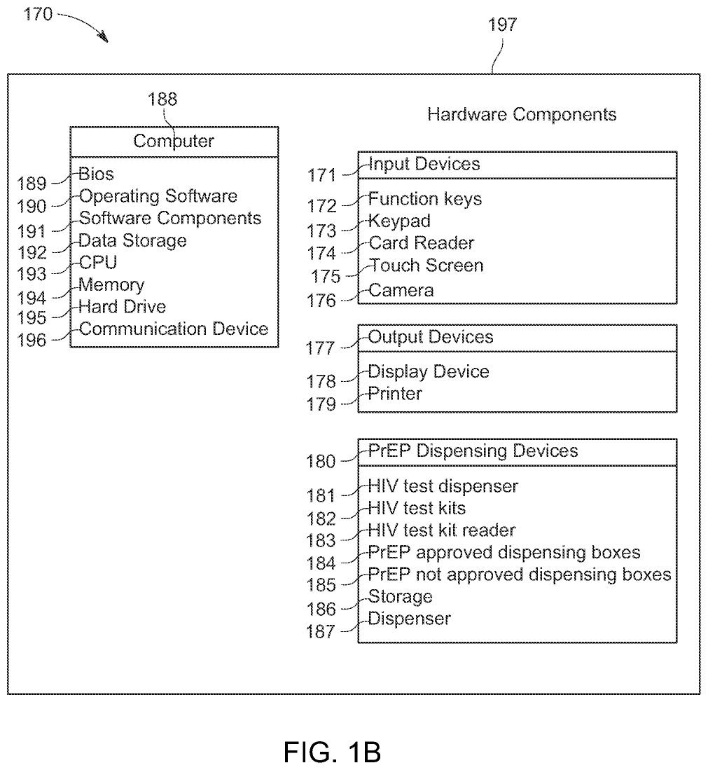

Referring now to FIG. 1A, FIG. 1A depicts an exemplary embodiment of a PrEP dispenser system 100. Three components of system 100 are PrEP dispenser 104, user device 108, and provider device 112. PrEP dispenser 104, user device 108, and provider device 112 form a network and are all in communication with each other through communication pathways 116, 120, and 124. Communication pathway 116 facilitates communication between PrEP dispenser 104 and provider device 112. Communication pathway 120 facilitates communication between PrEP provider device 112 and user device 108. Communication pathway 124 facilitates communication between PrEP dispenser 104 and user device 108. Each communication pathway 116, 120, and 124 are independent from each other and each use an appropriate communication method. Appropriate communication methods include wired and wireless connections, Blue Tooth connections, Wi-Fi connections, internet connections, cellular signal connections, and ethernet connections. In preferred embodiments, communication pathway 124 between user device 108 and PrEP dispenser is a Blue Tooth or cellular connection; communication pathway 120 between user device 108 and provider device 112 is a cellular connection; and communication pathway 116 between PrEP dispenser 104 and provider device 112 is an internet-based pathway or cellular connection. In some embodiments, each of communication pathways 116, 120, and 124 use a combination of communication methods to form a single communication pathway, for example, a communication pathway may include a WIFI connection between PrEP dispenser 104 and a WIFI router; and wired ethernet connection between the WIFI router and a cable internet modem; a wired connection from the cable internet modem to an internet service provider; and an internet data connection between a cellular service provider and provider device 112. Each communication pathway 116, 120, and 124 is capable of sending various forms of data between the network connected components of system 100. In a preferred embodiment, communication pathways 116, 120, and 124 are encrypted communication pathways.

In some embodiments, user device 108 and provider device 112 are both cellular smart phones. In other embodiments, user device 108 and provider device 112 are other types of devices capable of receiving and displaying data such as a general purpose computer or a tablet. User device 108 and provider device 112 need not be the same type of device. For example, in some embodiments user device 108 is a cellular smart phone and provider device 112 is a general purpose computer. Each of user device 108 and provider device 112 is capable of receiving and transmitting data, accepting user or provider input, and displaying data to the user or provider, respectively. In preferred embodiments, user device 108 and provider device 112 are both cellular smart phones and user and provider interaction is provided through a specific application operating on user device 108 and provider device 112. In some embodiments, there is no user device 108. In these embodiments, interaction with user 148 is accomplished through the user interaction module of PrEP dispenser 100.

In the embodiment of FIG. 1A, PrEP dispenser 104 is in the form of a kiosk, booth, or vending machine, with housing 197. In the depicted embodiment, PrEP dispenser 104 has a display screen 128, user identification module 132, user input device 136, dispenser 140, and trash receptacle 144. Display screen 128 is screen capable of display prompts and information to user 148 and, in some embodiments, is a touch screen capable of accepting user 148 input. User input 136 is any device capable of accepting input from user 148, and can include a keyboard, mouse, buttons, and the like or a combination of the same. User input 136 and display screen 128, if display screen 128 is a touch screen, make up the user interaction module of PrEP dispenser 100. Dispenser 140 is a module capable of dispensing PrEP dosages, HIV test kits, condoms, lubricants, etc., which are stored inside PrEP dispenser 104, to user 148. In some embodiments, PrEP dispenser 100 has a single dispenser 140 for dispensing all items to user 148. In other embodiments, PrEP dispenser 100 has multiple dispensers for individually dispensing different items to user 148. Identification module 132, in this embodiment, includes camera 156 and identification reader 160. Camera 156 is capable of taking photographs and/or videos of user 148 interacting with PrEP dispenser 104. Identification reader 160 is capable of reading, scanning, and/or imaging user 148 identification, such as a user's driver's license. HIV test reader 144 is capable of receiving, reading, and imaging HIV tests. Trash receptacle 152 is a secure trash receptacle for a user to dispose of any trash with or without sensitive or private user information.

In other embodiments, PrEP dispenser 104 may have a plurality of input devices such as touch screens, keypads, function keys, card readers, bar card readers or any other device that is operative to provide the dispenser with inputs representative of user instructions, information, or responses to prompts. PrEP dispenser 104 may also include one or more biometric input devices such as a fingerprint scanner, an iris scanner, facial recognition device, hand scanner, or any other biometric reading device which may be used to read a biometric input that can be used to identify a user. Collectively, these devices form a user identification module of PrEP dispenser 100.

In some embodiments, a video is displayed to user 148 on display screen 128 which provides instructions for administration of the HIV test kit dispensed from dispenser 140 or provides PrEP dosage instructions.

In some embodiments, PrEP dispenser 104 further includes a credit card reader (not shown) for accepting payment from user 148. In some embodiments, identification reader 160 is a combination identification reader and credit card reader.

Referring now to FIG. 1B, FIG. 1B is a schematic view of an embodiment of an interactive PrEP dispenser 170 showing components included in or in communication with an interactive PrEP dispenser 170. In this embodiment the input devices 171, output devices 177, PrEP dispensing devices 180, and computer 188 are installed in or onto and supported by housing 197.

Possible input devices 171 include function keys 172, keypad 173, card reader 174, camera 176 and touch screen 175. Input devices can be used by a user to input information and to respond to prompts to the user from PrEP dispenser 170. Input devices 171 are in communication with computer 188 to transmit information to computer 188.

Possible output devices 177 include display device 178 and printer 179. Output devices can be used to communicate prompts for information or transfer of information to a user of PrEP dispenser 170. Output devices 177 are in communication with computer 188 to transmit information from computer 188 to output devices 177.

PrEP dispensing devices 180 are the devices that are used in making a determination of whether or not PrEP should be dispensed to a user and for dispensing products to the user. PrEP dispensing devices 180 include HIV test kit dispenser 181, which is capable of dispensing HIV test kit 182 to a user who is requesting PrEP. Dispensing devices 180 also include, HIV test reader 183, which is capable of imaging an HIV test swab of HIV test kit 182 after an user self-administers an HIV test kit 182.

PrEP dispensing devices 180 also include devices used to dispense PrEP, information, lubricant, and/or condoms to a user after a determination of whether or not PrEP should be dispensed to the user. These include PrEP approved dispensing boxes 184, which contain, at least a PrEP prescription and PrEP dosage and usage instructions. The PrEP prescription may be maintenance dosage prescription or an on demand dosage prescription. PrEP approved dispensing boxes 184 may also contain condoms, lubricant, and additional information for the user. These also include PrEP not approved dispensing boxes 185, which do not contain a PrEP prescription, but may contain condoms, lubricant, and additional information for the user. In addition, PrEP dispensing devices 180 include storage compartments and transport mechanisms 186 for storing and delivering HIV test kits 182, PrEP approved dispensing boxes 184, and PrEP not approved dispensing boxes 186 to dispenser 187. Dispenser 187 is used to dispense items such as HIV test kits 182, PrEP approved dispensing boxes 184, and PrEP not approved dispensing boxes 186 to a user. PrEP dispensing devices 180 are in communication with computer 188 and are operated by or in response to signals and commands from computer 188 to PrEP dispensing devices 180.

Computer 188 may be a general-purpose computer containing bios 189, operating software 190, software components 191, data storage 192, central processing unit 193, memory 194, hard drive 195, and communication device 196. Computer 188 is used to process a request for PrEP from a user, control of input components 171, output devices 177, and PrEP dispensing devices 180 and is in operative communication with all of these devices of PrEP dispenser 170. Computer 188, through communication device 196, is in further operative communication with external devices, for example, such as user device 108 and provider device 112 of the embodiment of FIG. 1A.

In some embodiments, PrEP dispenser 104 or 170 is located in a location where a user 148 is likely to make a spontaneous decision to engage in sexual activity that has a potential to result in the transmission of HIV. In some embodiments, PrEP dispenser 104 or 170 is located in a bar, park, spa, gym, or other common meeting area for gay men.

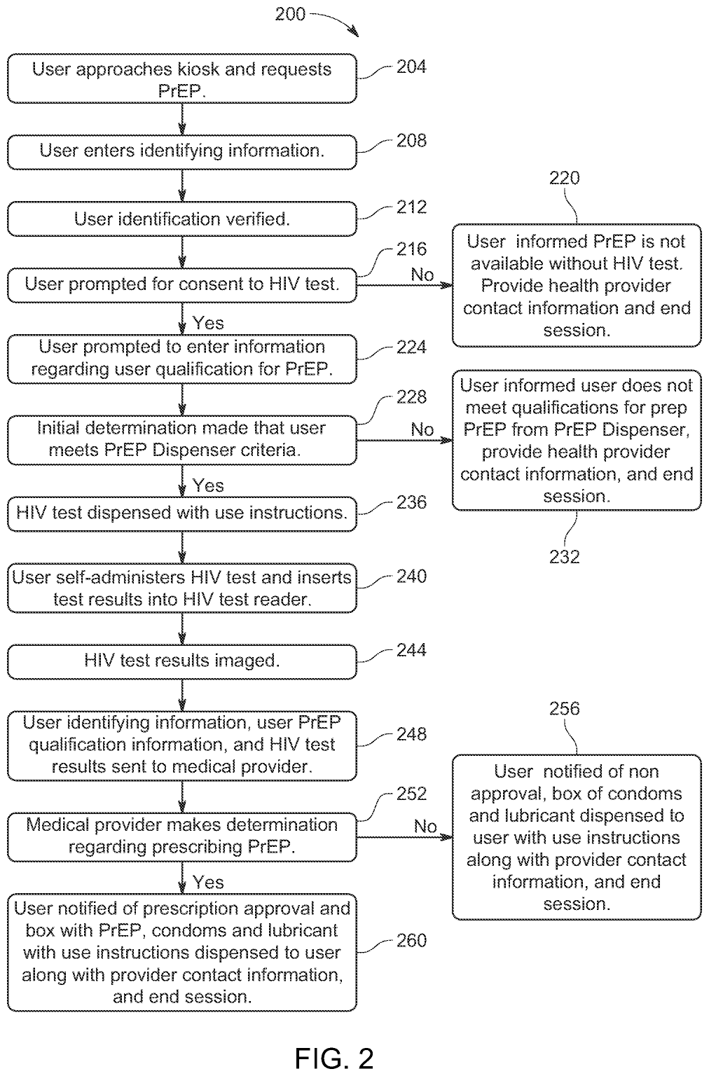

Referring now to FIG. 2, FIG. 2 depicts a flow chart for an exemplary embodiment of a method 200 for use of an interactive PrEP dispenser system or PrEP dispenser, such as the embodiments described in reference to FIG. 1A and FIG. 1B.

In step 204 user 148 who desires to acquire PrEP approaches PrEP dispenser 104. The user 148 is prompted on display screen 128 or user device 108 to enter user identifying information. User identifying information in some embodiments includes the user's name; a unique user identifier, such as an account number; a security pin; the user's driver's license number; an image of the user's driver's license; an image or video of the user, taken on camera 156 or a camera of user device 108; a reading of the user's fingerprint taken on a fingerprint imager of the user's device, or other identifying information.

Once user 148 enters the requested user information, the method proceeds to step 208. In step 208, the user identifying information is used to verify the identity of the user.

Once the identity of user 148 is confirmed, the method proceeds to step 216. In step 216, user 148 is prompted to consent to an HIV test. User 148 inputs either agreement or disagreement to consent to HIV testing. If user 148 does not consent to HIV testing, the process proceeds to step 220. In step 220, user 148 is notified that PrEP is not available without a negative HIV test, and user 148 is provided with a list of health providers and their contact information. The health provider information can be provided in print, dispensed from PrEP dispenser 104, or electronically, through user device 108. If user 148 consents to HIV testing, method 200 proceeds to step 224.

In step 224, user 148 is prompted to enter information regarding user PrEP qualification information for prescribing PrEP. User PrEP qualification information includes, at least, determining if user 148 is HIV positive, if user 148 has Hepatitis B, and if user 148 has a kidney disease. User 148 is prompted with each question and the option to provide a simple yes or no response. A yes response to any of these inquiries indicates that user 148 is not qualified for a PrEP prescription.

Once user qualification information is received, method 200 proceeds to step 228, in which PrEP dispenser 104 processes the qualification information to make an initial screening determination of whether or not user 148 qualifies for a PrEP prescription. If it is determined that user 148 does not meet the qualifications for a PrEP prescription, method 200 proceeds to step 232. In step 232, user 148 is informed that user 148 does not qualify for a PrEP prescription and user 148 is provided with a list of health providers and their contact information. The health provider information can be provided in print, dispensed from PrEP dispenser 104, or electronically, through user device 108. If a determination is made that the user 148 is not initially disqualified from receiving a PrEP prescription, then method 200 proceeds to step 236.

In step 236, PrEP dispenser 104 dispenses an HIV test kit to user 148 through dispenser 140. Instructions on use and self-administration of the HIV test is also provided to user 148. Instructions can be supplied to user 148 in paper with the HIV test kit, on display screen 128, and/or electronically through user device 108.

Method 200 then proceeds to step 240. In step 240, user 148 self-administers the HIV test and inserts the test results into HIV test reader 144. HIV test reader 144 processes and/or images the HIV test results in step 244. In some embodiments, the HIV test dispensed from PrEP dispenser 104 is a self-administered saliva-based test, such the Oraquick HIV Self-Test produced by OraSure Technologies, Inc. HIV test results are indicated by a visual indicator. HIV test reader 144, in this embodiment, is configured so that the test stick fits into a receiving slot of HIV test reader 144 so that HIV test reader 144 may image the test results through a camera of HIV test reader to create HIV test result information.

Method 200 then proceeds to step 248. In step 248, PrEP dispenser transmits user PrEP qualification information, user identification information, and HIV test result information to provider 164 through provider device 112.

Method 200 then proceeds to step 252. In step 252 provider 164 analyzes the information sent to provider 164 to determine if PrEP should be prescribed to user 148. Provider, through provider device 112, either prescribes PrEP to user 148 or declines to prescribe PrEP to user 148, which information is transmitted to PrEP dispenser 104.

If provider 164 did not prescribed PrEP to user 148, then method 200 proceeds to step 256. In step 256 user 148 is notified that PrEP has not been prescribed to user 148, and a box is dispensed from PrEP dispenser 104. Since PrEP has not been prescribed in this case, the box does not contain PrEP medication. Instead the box contains information regarding the spread of HIV and other sexually transmitted diseases; condoms; sex lubricant; and/or a list of health care providers and contact information.

If provider 164 did prescribe PrEP to user 148, then method 200 proceeds to step 260. In step 260 user 148 is notified that PrEP has been prescribed to user 148 and a box is dispensed from PrEP dispenser 104. Since PrEP has been prescribed to user 148, the box contains PrEP medication and dosage and other information regarding PrEP medication. The box can also contain information regarding the spread of HIV and other sexually transmitted diseases; condoms; sex lubricant; and/or a list of health care providers and contact information.

In some embodiments, the box dispensed in step 256 and the box dispensed in step 260 have an identical outer appearance. Doing so protects user 148 confidential information that may be deduced from an observer in a situation where user 148 receives a different response or box based on whether or not PrEP has been prescribed to user 148. For example, if PrEP was not prescribed and a box was not dispensed to user 148, or a box identical in outer appearance to one containing PrEP was not dispensed to user 148 then an observer may be able to deduce that user 148 is HIV positive.

In embodiments of PrEP dispenser system 100 and method 200, information including data, prompts, requests, results, etc. may be sent directly between user device 108 and provider device 112. In other embodiments such information may be sent to and from user device 108 and provider device 112 through PrEP dispenser 104.

In some embodiments, one or more steps of method 200 are performed by a computer of the PrEP dispenser 104, the user device 108, and/or the provider device 112. In some embodiments, one or more steps of method 200 are performed by a computer that is in network communication with PrEP dispensing system 100 such as a web based or cloud based computer. 1

Although the present invention and its advantages have been disclosed in the context of certain illustrative, non-limiting embodiments, it should be understood that various changes, substitutions, permutations, and alterations can be made without departing from the scope of the invention as defined by the claims. It will be appreciated that any feature that is described in a connection to any one embodiment may also be applicable to any other embodiment.

What is claimed:

1. A system for dispensing of PrEP comprising:
a kiosk for dispensing PrEP, wherein the kiosk comprises:
at least one dispenser, a user identification module, a user interaction module, and an HIV test reader;
a user device, for use by a user requesting a prescription for PrEP; and
a provider device, for use by a medical provider who is authorized to prescribed PrEP;
wherein the user device, the provider device, and the kiosk are each in network communication with each other;
wherein the user inputs user identification information and PrEP qualification information to the system through the user interaction module or the user device;
wherein the medical provider enters prescription information into the system through the provider device;
wherein the HIV reader receives and creates imaged HIV test results;
wherein the imaged HIV tests results, user information, and PrEP qualification information is transmitted to the provider device from the kiosk and the prescription information is transmitted to the kiosk from the provider device;
wherein a box is dispensed to the user from the at least one dispenser in response to the prescription information.

2. The system of claim 1 wherein, the user device and the provider device are cellular phones.

3. The system of claim 1 wherein, the dispenser dispenses an HIV test to the user in response to a user's consent to HIV testing.

4. The system of claim 1 wherein, the box contains PrEP medication if the prescribing information indicates that the medical provider has prescribed PrEP to the user or the box does not contain PrEP medication if the prescribing information indicates that the medical provider has not prescribed PrEP to the user; wherein the box containing PrEP and the box not containing PrEP are identical in outer appearance.

5. The system of claim 4 wherein the PrEP medication is an on demand PrEP dosage.

6. The system of claim 1 wherein the user identification module comprises at least one of a camera, an identification reader, a fingerprint scanner, or facial recognition device.

7. The system of claim 1 wherein the at least one dispenser comprises a single dispenser for dispensing the box and an HIV test kit.

8. The system of claim 1 wherein the at least one dispenser comprises a first dispenser for dispensing the box and a second dispenser for dispensing an HIV test kit.

9. The system of claim 1 wherein the network communication between the user device, the provider device, and the kiosk are encrypted communication pathways.

10. A method for interactive PrEP dispensing comprising the steps of:
receiving into a PrEP dispensing system user identification information and user PrEP qualification information;
verification of user identification by the PrEP dispensing system;
dispensing from a PrEP dispenser an HIV test to a user;
receiving and imaging by the PrEP dispenser an HIV test result;
transmitting the user identification information, user PrEP qualification information, and an imaged HIV test result to a medical provider through a network connection;
transmitting from the medical provider to the PrEP dispenser prescribing information; and
in response to the received prescribing information, dispensing from the PrEP dispenser either a box containing PrEP medication or a box not containing PrEP.

11. The method of claim 10 wherein the PrEP medication is an on demand PrEP dosage.

12. An interactive PrEP dispenser comprising:
a display screen for displaying information to a user;
one or more input devices for receiving information from a user;
a computer in network communication with a user device and a provider device;
an HIV test reader;
internal storage compartments for storing HIV test kits; boxes containing PrEP; and
boxes not containing PrEP, where the boxes containing PrEP and the boxes not containing PrEP have identical outer appearances;
at least one dispenser for dispensing the HIV tests kits, the boxes containing PrEP, and the boxes not containing PrEP.

13. The PrEP dispenser of claim 12 wherein the display screen is a touch screen capable of receiving input from a user.

14. The PrEP dispenser of claim 12 wherein at least one of the one or more input devices is one of a touch screen, buttons, or camera.

15. The PrEP dispenser of claim 13 wherein the display screen, the one or more input devices, the computer, the HIV test reader, and the internal storage compartments are installed in or onto and supported by a housing.

16. The PrEP dispenser of claim 15 wherein the housing is in the form of a kiosk.

17. The PrEP dispenser of claim 12 further comprising a user identification module.

18. The PrEP dispenser of claim 17 wherein the user identification module comprises at least one of a camera, an identification reader, a fingerprint scanner, or facial recognition device.

19. The PrEP dispenser of claim 12 wherein the boxes containing PrEP and the boxes not containing PrEP are identical in outward appearance.

20. The PrEP dispenser of claim 12 where in the boxes containing PrEP contain an on demand dosage of PrEP.
